(19) United States
(12) Patent Application Publication
(10) Pub. No.: US 2024/0307284 A1
(43) Pub. Date: Sep. 19, 2024

(54) EMULSION COMPOSITION AND PRODUCTION METHOD THEREOF

(71) Applicant: AJINOMOTO CO., INC., Tokyo (JP)

(72) Inventors: Kunitsugu TAKAHASHI, Tokyo (JP); Norikata KATSUKI, Tokyo (JP)

(73) Assignee: AJINOMOTO CO., INC., Tokyo (JP)

(21) Appl. No.: 18/658,553

(22) Filed: May 8, 2024

Related U.S. Application Data
(63) Continuation of application No. PCT/JP2022/041575, filed on Nov. 8, 2022.

(30) Foreign Application Priority Data
Nov. 9, 2021 (JP) 2021-182468

(51) Int. Cl. A61K 8/44; A61Q 19/00

(52) U.S. Cl. CPC A61K 8/442; A61Q 19/007; A61K 2800/21; A61K 2800/805

(57) ABSTRACT

Emulsion compositions containing (a) amino acid-based oil agent, (b) lecithin, (c) polyglycerin fatty acid ester, and (d) one or more kinds selected from the group consisting of polyaspartic acid and a salt thereof, and having a pH of 4.5 to 7, are superior in emulsion stability and pH stability and can penetrate between the cells of the stratum corneum layer of the skin.

### CROSS REFERENCES TO RELATED APPLICATIONS

This application is a continuation of International Patent Application No. PCT/JP2022/041575, filed on Nov. 8, 2022, and claims priority to Japanese Patent Application No. JP 2021-182468, filed on Nov. 9, 2021, both of which are incorporated herein by reference in their entireties.

### BACKGROUND OF THE INVENTION

### Field of the Invention

The present invention relates to emulsion compositions containing an amino acid-based oil agent, and production methods thereof.

### Discussion of the Background

In recent years, there has been a demand for cosmetics that can impart high moisturizing function and high barrier function to the skin. As components that can be expected to have both high moisturizing function and high barrier function, amino acid-based oil agents such as N-acyl amino acid esters and the like are known, and amino acid-based oil agents are generally applied to the skin as emulsion cosmetics like other oils.

However, most of general emulsion cosmetics contain emulsion having an average particle size of the order of microns, which is larger than the gap between the corneocytes of the skin. Oil droplets of this size only remain on the skin surface without penetrating into the gap between the corneocytes of the skin and are easily removed from the skin surface by friction with clothing, hand washing, and the like. Thus, it is difficult for them to impart a sustained moisturizing function to the skin.

As a method for solving such problems, the application of microemulsion technology has been considered. For example, a technique to provide emulsion type cosmetics that contain an amino acid-based oil agent in a specific range of content, lecithin in a specific range of content, polyhydric alcohol in a specific range of content, and water, and have an average particle size within the range of 30 nm to 200 nm (see JP-A-2006-089420, which is incorporated herein by reference in its entirety), and a technique to provide oil-in-water type microemulsions by using a surfactant whose main component is polyglycerin fatty acid ester with no carbon-carbon unsaturated bonds in the molecule, and glycerin and/or 1,3-butylene glycol contained in both the oil phase and the aqueous phase (see JP-A-2007-077077, which is incorporated herein by reference in its entirety) have been proposed.

However, the microemulsions described in JP-A-2006-089420 and JP-A-2007-077077 cannot be said to exhibit sufficient pH stability.

Therefore, there is a demand for an emulsion composition containing an amino acid-based oil agent, which is superior not only in emulsion stability but also pH stability and which can penetrate between the cells of the stratum corneum layer of the skin.

### SUMMARY OF THE INVENTION

Accordingly, it is an object of the present invention to provide an emulsion composition containing an amino acid-based oil agent, which is superior in emulsion stability and pH stability and which can penetrate between the cells of the stratum corneum layer of the skin.

This and other objects, which will become apparent during the following detailed description, have been achieved by the present inventors discovery that an emulsion composition superior in emulsion stability and pH stability even when it is a fine emulsion composition can be obtained by providing an emulsion composition containing (a) amino acid-based oil agent, (b) lecithin, (c) polyglycerin fatty acid ester, and (d) one or more kinds selected from the group consisting of polyaspartic acid and a salt thereof, and having a pH of 4.5 to 7.

Accordingly, the present invention provides the following.

(1) An emulsion composition comprising (a) amino acid-based oil agent, (b) lecithin, (c) polyglycerin fatty acid ester, and (d) one or more kinds selected from the group consisting of polyaspartic acid and a salt thereof, and having a pH of 4.5 to 7.

(2) The emulsion composition of (1), wherein the (a) amino acid-based oil agent is an ester of N-acylamino acid.

(3) The emulsion composition of (1) or (2), wherein the (b) lecithin is hydrogenated lecithin.

(4) The emulsion composition of any of (1) to (3), wherein the (c) polyglycerin fatty acid ester is a polyglycerin fatty acid ester with an HLB value of 14 to 17.

(5) The emulsion composition of any of (1) to (4), further comprising (e) water-soluble skin conditioning agent.

(6) The emulsion composition of (5), wherein the (e) water-soluble skin conditioning agent is one or more kinds selected from the group consisting of polyhydric alcohol, trimethylglycine, lactic acid and a salt thereof, pyrrolidonecarboxylic acid and a salt thereof, and amino acid and a salt thereof.

(7) The emulsion composition of any of (1) to (6), comprising emulsion particles with an average particle size of 30 nm to 500 nm.

(8) A method for producing an emulsion composition, comprising a step of uniformly mixing an oil phase component comprising (a) amino acid-based oil agent, (b) lecithin, and (c) polyglycerin fatty acid ester, adding an aqueous phase component comprising (d) one or more kinds selected from the group consisting of polyaspartic acid and a salt thereof to the aforementioned oil phase component, and mixing them, and a step of emulsifying under high-pressure the mixture obtained by the aforementioned step, wherein emulsion particles have an average particle size of 30 nm to 500 nm.

(9) The production method of (8), wherein the (a) amino acid-based oil agent is an ester of N-acylamino acid.

(10) The production method of (8) or (9), wherein the (b) lecithin is hydrogenated lecithin.

(11) The production method of any of (8) to (10), wherein the (c) polyglycerin fatty acid ester is a polyglycerin fatty acid ester with an HLB value of 14 to 17.

(12) The production method of any of (8) to (11), further comprising adding (e) water-soluble skin conditioning agent to the aqueous phase component.

(13) The production method of (12), wherein the (e) water-soluble skin conditioning agent is one or more kinds selected from the group consisting of polyhydric alcohol, trimethylglycine, lactic acid and a salt thereof, pyrrolidonecarboxylic acid and a salt thereof, and amino acid and a salt thereof.

(14) The production method of any of (8) to (13), comprising setting the pH of the emulsion composition to 4.5 to 7.

(15) A method for producing an emulsion composition, comprising a step of adding a part of water to an oil phase component comprising (a) amino acid-based oil agent, (b) lecithin, and (c) polyglycerin fatty acid ester and mixing to emulsify them under high pressure, and a step of mixing (d) one or more kinds selected from the group consisting of polyaspartic acid and a salt thereof with the rest of water and adding the mixture to the emulsion obtained in the aforementioned step, wherein emulsion particles have an average particle size of 30 nm to 500 nm.

(16) The production method of (15), wherein the (a) amino acid-based oil agent is an ester of N-acylamino acid.

(17) The production method of (15) or (16), wherein the (b) lecithin is hydrogenated lecithin.

(18) The production method of any of (15) to (17), wherein the (c) polyglycerin fatty acid ester is a polyglycerin fatty acid ester with an HLB value of 14 to 17.

(19) The production method of any of (15) to (18), further comprising adding polyhydric alcohol as the (e) water-soluble skin conditioning agent to a part of water to be added to the oil phase component.

(20) The production method of any of (15) to (19), wherein one or more kinds selected from the group consisting of trimethylglycine, lactic acid and a salt thereof, pyrrolidonecarboxylic acid and a salt thereof, and amino acid and a salt thereof are added as the (e) water-soluble skin conditioning agent to the rest of water and mixed with the emulsion.

(21) The production method of any of (15) to (20), comprising setting the pH of the emulsion composition to 4.5 to 7.

### Advantageous Effects of Invention

According to the present invention, an emulsion composition containing an amino acid-based oil agent, which is superior in emulsion stability and pH stability can be provided, and further, a fine emulsion composition containing an amino acid-based oil agent, which is superior in emulsion stability and pH stability and which can penetrate between the cells of the stratum corneum layer of the skin can be provided.

In addition, according to the present invention, a method for producing a fine emulsion composition containing an amino acid-based oil agent, which is superior in emulsion stability and pH stability and which can penetrate between the cells of the stratum corneum layer of the skin can be provided.

The emulsion composition of the present invention or an emulsion composition produced by the production method of the present invention can favorably exert the moisturizing function and skin barrier function of an amino acid-based oil agent and impart superior moisturizing property and barrier effect to the skin.

### DETAILED DESCRIPTION OF THE PREFERRED EMBODIMENTS

The emulsion composition of the present invention contains (a) amino acid-based oil agent, (b) lecithin, (c) polyglycerin fatty acid ester, and (d) one or more kinds selected from the group consisting of polyaspartic acid and a salt thereof.

The amino acid-based oil agent contained as component (a) in the emulsion composition of the present invention is a highly-polar emollient having an amino acid residue in the structure thereof, and a preferred example is an ester of N-acylamino acid.

As the ester of N-acylamino acid, esters of N-acylsarcosines and lower alcohol such as isopropyl N-lauroyl sarcosinate and the like; esters of N-acyl-N-methyl-β-alanine and mixture of sterol and higher alcohol such as (phytosteryl/decyltetradecyl) N-myristoyl-N-methyl-β-alaninate and the like; esters of N-acyl-L-glutamic acid and mixture of sterol and higher alcohol such as di(phytosteryl/behenyl/2-octyldodecyl) N-lauroyl-L-glutamate, di(phytosteryl/2-octyldodecyl) N-lauroyl-L-glutamate, di(cholesteryl/behenyl/2-octyldodecyl) N-lauroyl-L-glutamate, di(cholesteryl/2-octyldodecyl) N-lauroyl-L-glutamate, and the like, and the like can be mentioned.

For the purpose of the present invention, esters of N-acyl-L-glutamic acid and mixture of sterol and higher alcohol are more preferably used, which can form lamella liquid crystal, similar to ceramides.

In the emulsion composition of the present invention, one kind of amino acid-based oil agent such as an ester of N-acylamino acid can be used alone, or two or more kinds thereof can also be used in combination.

In the present invention, the above-mentioned amino acid-based oil agent can also be synthesized by a known chemical synthesis method and used, and commercially available products provided by Ajinomoto Co., Inc., etc. can also be used.

The content of component (a) in the emulsion composition of the present invention is preferably 0.5 wt % to 10 wt %, more preferably 1 wt % to 10 wt %.

Lecithin contained as component (b) in the emulsion composition of the present invention is a lipid mixture containing phospholipid such as phosphatidyl choline, and examples include soybean lecithin obtained from soybean and egg yolk lecithin obtained from egg yolk.

From the aspects of oxidative stability of the emulsion composition, it is preferable to use hydrogenated lecithin such as hydrogenated soybean phospholipid and hydrogenated egg yolk lecithin.

In the emulsion composition of the present invention, one kind of lecithin can be used alone, or two or more kinds thereof can also be used in combination.

In the present invention, the above-mentioned lecithin can also be extracted from soybean, egg yolk, and the like and purified for use, and commercially available products provided by each company can also be used.

The content of component (b) in the emulsion composition of the present invention is preferably 0.06 wt % to 1.5 wt %, more preferably 0.1 wt % to 1.2 wt %.

A polyglycerin fatty acid ester contained in the emulsion composition of the present invention as component (C) is an ester of glycerin polymer and fatty acid. For the object of the present invention, hydrophilic polyglycerin fatty acid esters such as monoesters of polyglycerin having an average degree of polymerization of glycerin of around 6 to 10 and fatty acid having a carbon number of around 12 to 18, and the like are preferred, and polyglycerin fatty acid esters having a HLB (Hydrophilic-Lipophilic Balance) value of 14 to 17 are more preferred.

Examples of the polyglycerin fatty acid ester include hexaglyceryl monolaurate, decaglyceryl monolaurate, decaglyceryl monomyristate, decaglyceryl monostearate, and the like.

In the emulsion composition of the present invention, one kind of polyglycerin fatty acid ester can be used alone, or two or more kinds thereof can also be used in combination.

In the present invention, the above-mentioned polyglycerin fatty acid ester can also be chemically synthesized and used according to known production methods, and commercially available products provided by each company can also be used.

The content of component (c) in the emulsion composition of the present invention is preferably 0.04 wt % to 1 wt %, more preferably 0.08 wt % to 0.8 wt %.

The polyaspartic acid contained as component (d) in the emulsion composition of the present invention is L-aspartic acid, D-aspartic acid or DL-aspartic acid polymerized by an α-amide bond or a β-amide bond.

For the purpose of the present invention, poly L-aspartic acid and poly DL-aspartic acid are preferably used, and poly L-aspartic acid is more preferably used. In addition, any of an α-amide conjugate, a β-amide conjugate, and a mixture of an α-amide conjugate and a β-amide conjugate can be used, and an α-amide conjugate and a mixture of an x-amide conjugate and a β-amide conjugate are preferably used.

In the present invention, polyaspartic acid with a weight average molecular weight of 1,000 to 100,000 as measured by gel filtration chromatography is used. Polyaspartic acid with the aforementioned weight average molecular weight of 2,000 to 50,000 is preferably used, 2,000 to 10,000 is more preferably used, and 3,000 to 5,000 is further preferably used.

In the present invention, a salt of the above-mentioned polyaspartic acid can also be used as component (d).

The salt of polyaspartic acid is not particularly limited as long as it is a pharmaceutically acceptable salt and used for skin compositions. Examples thereof include salts with inorganic bases such as alkali metal salts (e.g., lithium salt, sodium salt, potassium salt, and the like); alkaline earth metal salts (e.g., magnesium salt, calcium salt, and the like); ammonium salt; and the like, and salts with organic bases such as alkanol amine salts (e.g., mono ethanolamine salt, diethanolamine salt, triethanolamine salt, and the like); salts with basic amino acids (e.g., arginine salt, lysine salt, histidine salt, and the like); and the like, and alkali metal salts such as sodium salt and the like are preferably used.

Polyaspartic acid and a salt thereof can be produced by a known method.

For example, a mixture of an α-amide conjugate and a β-amide conjugate can be obtained by a solid state polymerization method including directly heating and condensing amino acid (The Journal of the American Chemical Society 82 3745 (1960), which is incorporated herein by reference in its entirety, etc.).

An α-amide conjugate can be obtained by a method of ring opening the N-carboxylic acid anhydride of an amino acid and subjecting same to decarboxylation polymerization (Journal of Polymer Science A14 2065 (1976) and JP-B-s46-27828, which are incorporated herein by reference in their entireties, etc.), a method of polymerizing an active amino acid ester (JP-A-s54-47799), which is incorporated herein by reference in its entirety), a method of thermally polymerizing an N-dithiocarbonylalkoxycarbonylamino acid (JP-B-45-9391), which is incorporated herein by reference in its entirety), and the like.

In addition, a polymer that retains the optical activity of aspartic acid used as a starting material can be obtained by ring-opening polymerization of N-carboxylic acid anhydride of amino acid.

In the present invention, polyaspartic acid or a salt thereof may be produced and used by the above-mentioned known methods. Alternatively, commercially available products provided by Ajinomoto Co., Inc., etc. can also be used.

In the emulsion composition of the present invention, one kind selected from the group consisting of the above-mentioned polyaspartic acid and a salt thereof may be used alone, or two or more kinds thereof may be selected and used in combination.

The content of component (d) in the emulsion composition of the present invention is preferably 0.008 wt % to 0.85 wt %, more preferably 0.12 wt % to 0.5 wt %.

When a polyaspartic acid salt is contained as component (d), the above-mentioned content is shown as a content converted to that of a free form.

From the aspect of the emulsion stability of the emulsion composition, the content ratio of component (a) to component (b), that is, (a)/(b), in the emulsion composition of the present invention is preferably 0.33 to 166.7, more preferably 0.83 to 100, in weight ratio.

In addition, the content ratio of component (a) to component (c), that is, (a)/(c), in the emulsion composition of the present invention is preferably 0.5 to 250, more preferably 1.25 to 125, in weight ratio.

Further, the content ratio of component (a) to component (d), that is, (a)/(d), in the emulsion composition of the present invention is preferably 0.58 to 1250, more preferably 2 to 83.4, in weight ratio.

Furthermore, the content ratio of component (d) to the total of the content of component (a), the content of component (b), and the content of component (c), that is, (d)/[(a)+(b)+(c)], in the emulsion composition of the present invention is preferably 0.0006 to 1.42, more preferably 0.01 to 0.43, in weight ratio.

For the amino acid-based oil agent as component (a) to favorably exhibit the moisturizing function and barrier function of the skin, the total of the content of component (a), the content of component (b), the content of component (c) and the content of component (d), that is, (a)+(b)+(c)+(d), to the total amount of the emulsion composition is, preferably 0.61 wt % to 13 wt %, more preferably 1.3 wt % to 12.5 wt %.

The emulsion composition of the present invention may contain, in addition to the above-mentioned component (a) to component (d), (e) water-soluble skin conditioning agent.

In the present specification, the “water-soluble skin conditioning agent” refers to a water-soluble component that can improve dry or damaged skin, which is a water-soluble moisturizer and a water-soluble component having functions such as skin barrier function-improving effect, anti-inflammatory/anti-allergic action, antioxidant action, cell activation action, whitening action, and anti-aging action and the like.

As the water-soluble skin conditioning agent contained as component (e) in the emulsion composition of the present invention, polyhydric alcohol, trimethylglycine, lactic acid and a salt thereof, pyrrolidonecarboxylic acid and a salt thereof, amino acid and a salt thereof and the like can be mentioned.

As the polyhydric alcohol, divalent alcohols such as 1,2-propanediol, 1,3-butanediol, 1,2-hexanediol, dipropylene glycol and the like; trivalent alcohols such as glycerin and the like; sugar alcohols such as xylitol, sorbitol, maltitol and the like, and the like can be mentioned, and 1,2-propanediol, 1,3-butanediol, 1,2-hexanediol, dipropylene glycol, glycerin, and the like are preferably used.

In the present invention, polyhydric alcohol can be produced by a chemical synthesis method and the like and used, and commercially available products provided by each company can also be used.

Trimethylglycine is a natural amino acid derivative extracted from molasses, which is a byproduct of beet sugar (sugar beet sugar) and can be extracted from beet sugar (sugar beet sugar), purified and used, and commercially available products provided by each company can also be used.

As lactic acid (2-hydroxypropanoic acid), any of the L-form, D-form, and DL-form can be used. L-form and DL-form are preferably used, and L-form is more preferably used.

As the salt of lactic acid, salts with inorganic base such as alkali metal salts such as sodium lactate, potassium lactate, and the like; alkaline earth metal salts such as magnesium lactate, calcium lactate, and the like; ammonium lactate, and the like, and salts with organic base such as alkanol amine salts such as triethanolamine lactate and the like, and the like can be mentioned.

For the purpose of the present invention, an alkali metal salt of lactic acid is preferably used, and a sodium salt is more preferably used.

In the present invention, the above-mentioned lactic acid and a salt thereof can be produced by an extraction from animals and plants and purification method, a chemical synthesis method, a fermentation method, a gene recombinant method or the like and used, and commercially available products provided by each company can also be used.

As pyrrolidonecarboxylic acid (2-keto-pyrrolidine-5-carboxylic acid), any of D-pyrrolidonecarboxylic acid, L-pyrrolidonecarboxylic acid, DL-pyrrolidonecarboxylic acid may be used, and preferably, L-pyrrolidonecarboxylic acid and DL-pyrrolidonecarboxylic acid can be used.

As the salt of pyrrolidonecarboxylic acid, salts with inorganic base such as alkali metal salts such as sodium salt, potassium salt, and the like, alkaline earth metal salts such as calcium salt, magnesium salt, and the like, zinc salts, ammonium salts, and the like; and salts with organic base such as alkanolamine salts such as ethanolamine salt, diethanolamine salt, and the like, salts with basic amino acid such as lysine salt, arginine salt, and the like, and the like can be mentioned.

For the purpose of the present invention, pyrrolidonecarboxylic acid is preferably used in a free form or in the form of an alkali metal salt, and more preferably used in a free form or in the form of a sodium salt.

Pyrrolidonecarboxylic acid and a salt thereof can be produced by a chemical synthetic method, an enzyme method, a gene recombinant method, and the like and used. Commercially available products provided by each company can also be used.

Examples of the amino acid include acidic amino acids such as glutamic acid (2-aminopentanedioic acid), aspartic acid (2-aminobutanedioic acid), and the like; basic amino acids such as arginine (2-amino-5-guanidinopentanoic acid), lysine (2,6-diamino hexanoic acid), histidine (2-amino-3-(1H-imidazol-4-yl) propionic acid), citrulline (2-amino-5-(carbamoylamino)pentanoic acid), ornithine (2,5-diamino pentanoic acid), and the like; and neutral amino acids such as aliphatic amino acids (glycine (aminoethanoic acid), alanine (2-aminopropanoic acid), valine (2-amino-3-methylbutanoic acid), leucine (2-amino-4-methylpentanoic acid), isoleucine (2-amino-3-methylpentanoic acid)), amino acids having a hydroxy group (serine (2-amino-3-hydroxypropanoic acid), threonine (2-amino-3-hydroxybutanoic acid)), amino acids containing sulfur (cysteine (2-amino-3-mercaptopropanoic acid), methionine (2-amino-4-(methylthio) butanoic acid)), amino acids having an amide group (asparagine (2-amino-3-carbamoylpropanoic acid), glutamine (2-amino-4-carbamoylbutanoic acid)), amino acids having an imino group (proline (pyrrolidine-2-carboxylic acid)), amino acids having an aromatic group (phenylalanine (2-amino-3-phenylpropanoic acid), tyrosine (2-amino-3-(4-hydroxyphenyl) propanoic acid), tryptophan (2-amino-3-(indolyl) propanoic acid)), and the like.

As the above-mentioned amino acid other than glycine, any of the D-form, L-form, and DL-form can be used. L-form and DL-form are preferably used, and L-form is more preferably used.

The salt of the above-mentioned amino acid is not particularly limited as long as it is a pharmaceutically acceptable salt and is used in a composition for skin. Examples thereof include acid addition salts and salts with bases.

Specifically, salts with inorganic bases, organic bases, inorganic acids, and organic acids, and salts with amino acids can be mentioned.

Examples of the salts with inorganic bases include salts with alkali metals such as lithium, sodium, potassium, and the like, salts with alkaline earth metals such as magnesium, calcium, and the like, ammonium salts, and the like.

Examples of the salts with organic bases include salts with alkanol amines such as mono ethanolamine, diethanolamine, triethanolamine, and the like, salt with heterocyclic amines such as morpholine, piperidine, and the like, and the like.

Examples of the salts with inorganic acids include salts with hydrohalic acid (hydrochloric acid, hydrobromic acid, hydroiodic acid, etc.), sulfuric acid, nitric acid, phosphoric acid, and the like.

Examples of the salts with organic acids include salts with monocarboxylic acids such as formic acid, acetic acid, propanoic acid, and the like; salts with saturated dicarboxylic acids such as oxalic acid, malonic acid, succinic acid, and the like; salts with unsaturated dicarboxylic acids such as maleic acid, fumaric acid, and the like; salts with tricarboxylic acids such as citric acid and the like; salts with keto acids such as α-ketoglutaric acid and the like, and the like.

Examples of the salts with amino acids include salts with aliphatic amino acids such as alanine and the like; salts with aromatic amino acids such as tyrosine and the like; salts with basic amino acids such as lysine and the like; salts with acidic amino acids such as aspartic acid, glutamic acid, and the like, and the like.

The above-mentioned salts may be each a hydrate (hydrate salt), and examples of such hydrate include 1 hydrate to 6 hydrate.

In the present invention, the above-mentioned acidic amino acids are preferably used in the form of free forms and alkali metal salts such as sodium salt and potassium salt; the basic amino acids are preferably used in the form of free forms and acid addition salts such as hydrochlorides; and neutral amino acids are preferably used in the form of free forms.

The above-mentioned acidic amino acids and salts thereof, basic amino acids and salts thereof, and neutral amino acids and salts thereof to be used may be all extracted and purified from naturally occurring animals and plants, or obtained by chemical synthesis method, fermentation method, enzyme method, or gene recombinant method. Alternatively, commercially available products provided by each company may also be used.

In the emulsion composition of the present invention, one or more kinds selected from the group consisting of polyhydric alcohol, trimethylglycine, lactic acid and a salt thereof, pyrrolidonecarboxylic acid and a salt thereof, and amino acid and a salt thereof can be preferably used as component (e).

In order to impart the emulsion composition of the present invention with higher moisturizing function, it is preferable to use two or more kinds selected from the group consisting of amino acids and salts thereof, and more preferable to use one or more kinds selected from the group consisting of acidic amino acids and salts thereof, one or more kinds selected from the group consisting of basic amino acids and salts thereof, and one or more kinds selected from the group consisting of neutral amino acids and salts thereof in combination.

Furthermore, from the aspect of imparting a high moisturizing function, one or more kinds selected from the group consisting of acidic amino acid and a salt thereof, one or more kinds selected from the group consisting of basic amino acid and a salt thereof, and one or more kinds selected from the group consisting of neutral amino acid and a salt thereof are each preferably selected from amino acids and salts thereof constituting natural moisturizing factors.

That is, it is more preferable to use one or more kinds selected from the group consisting of aspartic acid, glutamic acid, and salts of these, one or more kinds selected from the group consisting of lysine, histidine, arginine, and salts of these, and one or more kinds selected from the group consisting of asparagine, threonine, serine, glutamine, glycine, alanine, valine, methionine, isoleucine, leucine, phenylalanine, tyrosine, proline, and salts of these in combination.

Furthermore, from the aspect of the moisturizing function to be imparted to the emulsion composition, as component (e), one or more kinds of polyhydric alcohol, trimethylglycine, one or more kinds selected from the group consisting of lactic acid and a salt thereof, one or more kinds selected from the group consisting of pyrrolidonecarboxylic acid and a salt thereof, and one or more kinds selected from the group consisting of amino acid and a salt thereof are preferably used in combination, one or more kinds of polyhydric alcohol, trimethylglycine, one or more kinds selected from the group consisting of lactic acid and salts thereof, one or more kinds selected from the group consisting of pyrrolidonecarboxylic acid and a salt thereof, and two or more kinds selected from the group consisting of amino acid and a salt thereof are more preferably used in combination, one or more kinds of polyhydric alcohol, trimethylglycine, one or more kinds selected from the group consisting of lactic acid and a salt thereof, one or more kinds selected from the group consisting of pyrrolidonecarboxylic acid and a salt thereof, one or more kinds selected from the group consisting of acidic amino acid and a salt thereof, one or more kinds selected from the group consisting of basic amino acid and a salt thereof, and one or more kinds selected from the group consisting of neutral amino acid and a salt thereof are further preferably used in combination, and one or more kinds of polyhydric alcohol, trimethylglycine, one or more kinds selected from the group consisting of lactic acid and a salt thereof, one or more kinds selected from the group consisting of pyrrolidonecarboxylic acid and a salt thereof, one or more kinds selected from the group consisting of aspartic acid, glutamic acid, and these salts, one or more kinds selected from the group consisting of lysine, histidine, arginine, and these salts, and one or more kinds selected from the group consisting of asparagine, threonine, serine, glutamine, glycine, alanine, valine, methionine, isoleucine, leucine, phenylalanine, tyrosine, proline, and these salts are even more preferably used in combination.

In the present invention, as trimethylglycine, one or more kinds selected from the group consisting of lactic acid and a salt thereof, one or more kinds selected from the group consisting of pyrrolidonecarboxylic acid and a salt thereof, and one or more kinds selected from the group consisting of amino acid and a salt thereof to be used as component (e), a previously-mixed commercially available product can also be used.

In the emulsion composition of the present invention, the content of component (e) is preferably 0.05 wt % to 4.5 wt %, more preferably 0.5 wt % to 4 wt %.

When lactate, pyrrolidonecarboxylic acid salt, and amino acid salt are contained as component (e), the contents thereof are converted into those of free forms, and the content of component (e) is calculated.

The emulsion composition of the present invention can contain, in addition to the above-mentioned components (a)-(e), general additives added to skin external preparations and cosmetics, to the extent not impairing the characteristics of the present invention.

As such additive, hydrophilic solvents such as water, lower alcohol (ethanol, isopropanol, etc.), and the like;

hydrophilic bases such as gelatin, polyethylene glycol, and the like; oleaginous bases such as animal and plant fats and oils (olive oil, soybean oil, camellia oil, sesame oil, peanuts oil, cacao butter, beef tallow, lard, etc.), waxes (carnauba wax, beeswax, jojoba oil, etc.), aliphatic alcohols (octyldodecanol, cetanol, stearyl alcohol etc.), fatty acids (oleic acid, palmitic acid, stearic acid, etc.), hydrocarbons (squalane, white petrolatum, liquid paraffin, ceresin, microcrystalline wax, etc.), and the like; suspending agents such as propylene glycol alginate, dioctyl sodium sulfosuccinate, povidone, and the like; surfactants or emulsifiers other than lecithin and polyglycerin fatty acid ester, such as polyoxyethylene hydrogenated castor oil, polyoxyethylene alkyl ether (polyoxyethylene oleyl ether etc.), polyoxyethylene sorbitan fatty acid ester (polyoxyethylene sorbitan monolaurate, polyoxyethylene sorbitan monostearate, polyoxyethylene sorbitan monooleate, etc.), glycerin fatty acid ester (glyceryl monostearate etc.), sucrose fatty acid ester, and the like; thickening agents such as carboxyvinyl polymer, sodium polyacrylate, xanthan gum, carboxymethylcellulose, hydroxypropylcellulose, poly(vinyl alcohol) (partially saponified product), and the like; stabilizers such as sodium edetate, sorbitol, thymol, polyoxyethylene polyoxypropylene glycol, and the like; antioxidants such as ascorbic acid, sodium erythorbate, tocopheryl acetate, dibutyl hydroxytoluene and the like; preservatives such as sorbic acid, sodium dehydroacetate, methyl p-hydroxybenzoate, propyl p-hydroxybenzoate, butyl p-hydroxybenzoate, phenoxyethanol, and the like; pH adjusters such as hydrochloric acid, citric acid, sodium citrate, acetic acid, sodium acetate, potassium hydroxide, sodium hydroxide, sodium hydrogenphosphate, and the like; dye; pigment; flavor, and the like can be mentioned.

Where necessary, one or more kinds of the above-mentioned additives can be used.

The pH of the emulsion composition of the present invention is 4.5 to 7, preferably 5 to 7. When the pH of the emulsion composition of the present invention on preparation is not within the aforementioned range, it is adjusted to 4.5 to 7 by the above-mentioned pH adjuster.

The pH of the emulsion composition of the present invention can be measured by the glass electrode method in accordance with the provisions of Japanese Industrial Standards (JIS) Z 8802:2011, 17th Edition Japanese Pharmacopoeia General Test Method 2.54 pH measurement method), which is incorporated herein by reference in its entirety, and the like.

The viscosity of the emulsion composition of the present invention is preferably 0.1 mPa·s to 1000 mPa·s, more preferably 0.1 mPa·s to 100 mPa·s, further preferably 0.1 mPa·s to 20 mPa·s.

The viscosity of the emulsion composition of the present invention can be calculated from the measurement value after rotating for 30 seconds at a rotation speed of 60 rpm at 25° C. using a Brookfield type viscometer (Toki Sangyo Co., Ltd, TVB-10 type, etc.), Rotor No. 1.

In order for the amino acid-based oil agent having a moisturizing function and a barrier function to penetrate well between the cells of the skin stratum corneum, the average particle size of the emulsion particle in the emulsion composition of the present invention is preferably 30 nm to 500 nm, more preferably 60 nm to 200 nm, further preferably 60 nm to 150 nm. The average particle size of the emulsion particles is measured by the dynamic light scattering method.

Specifically, the emulsion composition to be measured at 25° C. is appropriately diluted with purified water, and the scattering intensity is measured using dynamic light scattering by a particle size analyzer for concentrate using a semiconductor laser as a light source, for example, FPAR-1000 (Otsuka Electronics Co., Ltd.). From the obtained scattering intensity distribution, the average particle size can be determined by cumulant analysis. The emulsion composition may be diluted so that the amount of the oil phase component of the emulsion composition is 18 by weight to 10% by weight. By diluting the composition to fall within this range, the average particle size of the emulsion particles can be appropriately measured.

The emulsion composition of the present invention can be produced by a general emulsion method. For example, an oil phase component that is a mixture of the above-mentioned component (a) to component (c) and a lipophilic additive to be added as necessary, and an aqueous phase component in which the above-mentioned component (d), or component (d) and component (e), and a hydrophilic additive to be added as necessary are dissolved in water are each dissolved by warming or heating, the oil phase component and the aqueous phase component are mixed by stirring and emulsified.

In addition, it can also be produced by adding an aqueous phase component in which polyhydric alcohol from the above-mentioned component (e) is added to water and heated to an oil phase component in which a lipophilic additive to be added as necessary is added to the above-mentioned component (a) to component (c), and the mixture is dissolved by heating, mixing by stirring to emulsify the mixture, adding the above-mentioned component (d), component (e) other than polyhydric alcohol, and hydrophilic additive to be added as necessary, which are dissolved in water, and mixing them.

To afford a fine emulsion composition having the above-mentioned average particle size, it is preferable to emulsify using a high-pressure emulsification equipment such as high-pressure homogenizer, microfluidizer, ultra high-pressure wet-type atomization equipment and the like.

The emulsion composition of the present invention having an average particle size of emulsion particles of 30 nm to 500 nm is preferably produced by the below-mentioned production method.

The emulsion composition of the present invention can be preferably provided as a skin external preparation in the form of emulsion formulation, cream, and the like; skin cosmetics such as milky lotion, cream and the like; makeup base such as base toner, base cream and the like; makeup cosmetics such as emulsified liquid foundation, cream foundation and the like; body cosmetics such as body toner, body cream and the like; sunscreen cosmetics such as sunscreen milky lotion, sunscreen cream and the like; and the like.

The emulsion composition of the present invention contains an amino acid-based oil agent and is superior in emulsion stability and pH stability. Furthermore, the fine emulsion composition of the present invention can favorably penetrate between the cells of the skin stratum corneum.

The present invention can favorably exhibit a moisturizing function and a barrier function of the skin that an amino acid-based oil agent has.

The present invention also provides a production method of a fine emulsion composition containing an amino acid-based oil agent (hereinafter to be also referred to as “the production method of the present invention” in the present specification).

The production method of the present invention includes a step of uniformly mixing an oil phase component containing (a) amino acid-based oil agent, (b) lecithin, and (c) polyglycerin fatty acid ester, adding an aqueous phase component containing (d) one or more kinds selected from the group consisting of polyaspartic acid and a salt thereof, or (d) one or more kinds selected from the group consisting of polyaspartic acid and a salt thereof and (e) water-soluble skin conditioning agent, to the aforementioned oil phase component, and mixing them, and a step of emulsifying under high-pressure the mixture obtained by the aforementioned step.

Each of component (a) to component (e) is as described for the emulsion composition of the present invention.

In order to obtain a uniform oil phase by mixing an oil phase component, namely component (a) to component (c) and a lipophilic additive to be added as necessary, it is preferable to mix the aforementioned oil phase components and heat the mixture to about 70° C. to 85° C. It is also preferable to warm an aqueous phase component containing component (d), or component (d) and component (e), and a hydrophilic additive to be added as necessary to about 50° C. to 60° C. before mixing with the aforementioned oil phase.

Mixing of the oil phase and the aqueous phase can be performed using a general mixer such as a mixer with stirring blades or a homo mixer. Emulsification under high pressure is performed using a high-pressure emulsification equipment such as high-pressure homogenizer, microfluidizer, ultra high-pressure wet-type atomization equipment, and the like. Emulsifying under high pressure is performed at 50° C. to 90° C. preferably under pressurization at 100 MPa to 250 MPa, more preferably 120 MPa to 200 MPa.

Furthermore, the production method of the present invention preferably includes setting the pH of the emulsion composition to 4.5 to 7, more preferably pH 5 to 7.

The pH of the emulsion composition is adjusted by the above-mentioned pH adjuster according to a conventional method.

As another embodiment, the production method of the present invention includes a step of uniformly mixing an oil phase component containing component (a) to component (c), adding a part of water, mixing them, and emulsifying them under high pressure, and a step of mixing component (d) with the rest of water and adding the mixture to the emulsion obtained by the afore-mentioned step to make it uniform. Here, the step of mixing the oil phase and a part of the water, and the step of adding the component (d) mixed with the rest of the water to the obtained emulsion and uniformly mixing the same can be performed using the above-mentioned general mixer, and emulsification under high pressure is performed using above-mentioned high-pressure emulsification equipment.

When polyhydric alcohol is contained as component (e), polyhydric alcohol is preferably added to a part of water in the emulsifying step under high pressure. In addition, component (e) other than polyhydric alcohol and other aqueous phase components are preferably added together with component (d) and the rest of water.

In this embodiment, the oil phase and a part of water to be mixed in the emulsifying step under high pressure are each preferably heated and warmed in advance at about 70° C. to 85° C. and about 50° C.-60° C. The temperature and pressure when emulsifying under high pressure are the same as those in the emulsification under high pressure in the above-mentioned embodiment.

The production method in this embodiment preferably includes setting the pH of the emulsion composition to 4.5 to 7, more preferably 5 to 7. The adjustment of the pH of the emulsion composition is the same as that in the above-mentioned embodiment.

According to the production method of the present invention, a fine emulsion composition containing an amino acid-based oil agent can be obtained. The average particle size of the emulsion particles in the emulsion composition obtained by the production method of the present invention is preferably 30 nm to 500 nm, more preferably 60 nm to 200 nm, further preferably 60 nm to 150 nm.

The emulsion composition obtained by the production method of the present invention is superior in the emulsion stability and pH stability, can favorably penetrate between the cells of the skin stratum corneum, and can favorably exhibit the moisturizing function and barrier function that the amino acid-based oil agent has.

Other features of the invention will become apparent in the course of the following descriptions of exemplary embodiments which are given for illustration of the invention and are not intended to be limiting thereof.

### EXAMPLES

### Examples 1-10, Comparative Examples 1-9. Emulsion Cosmetics

In Table 1, components (1) to (3) (components (a), (b), (c)) were mixed, dissolved by heating to 70° C. to prepare a uniform oil phase, components (4) and (5) (component (e)) were dissolved in (6), warmed to 50° C. and mixed in a homomixer (“homomixer MARK II 2.5 type”, PRIMIX Corporation), and further subjected to a homogenization treatment by a high-pressure emulsification equipment (ultra high-pressure wet-type atomization equipment “Nanovater”, YOSHIDA KIKAI CO., LTD.) at pressure 150 MPa, temperature 90° C., whereby a fine emulsion was prepared. Then, components (7) to (22) (component (e), component (d) or alternative component of component (d), hydrophilic additive) were added to and dissolved in (23), and the resulting solution was added and mixed to prepare the emulsion cosmetics of Examples 1-10 and Comparative Examples 1 to 8.

Also, in the above-mentioned production method, emulsification under atmospheric pressure was performed using a homomixer (“homomixer MARK II 2.5 type”, PRIMIX Corporation) instead of the homogenization treatment by a high-pressure emulsification equipment, whereby the emulsion cosmetic of Comparative Example 9 was prepared.

As the components of (1) to (5) and (7) to (22), products commercially available for cosmetics were used. The weight average molecular weight measured by gel filtration chromatography of poly(sodium L-aspartate) of (19) was 3,000 to 5,000.

For the respective emulsion cosmetics of Examples 1 to 10 and Comparative Examples 1 to 9, as described below, the pH stability and emulsion stability were evaluated, and the average particle size of the emulsion particles of the emulsion cosmetics shown below was measured. The results are also shown in Table 1.

### (1) pH Stability

The pH was measured as follows using an F-74 table-top pH meter and a composite electrode 9615S-10D (Horiba, Ltd.).

The sensitivity was adjusted in advance at 25° C. at two points of pH=4.01 and 6.86, using a phthalate standard solution and a neutral phosphate standard solution specified by Japanese Industrial Standards (JIS), and the electrode sensitivity was set to 96% or more. Each of the emulsion cosmetics of Examples and Comparative Examples was measured at 25° C., the measured value at the time of preparation was compared with the measured value after storage at 50° C. for 2 months, and the pH stability was evaluated according to the following evaluation criteria.

### <Evaluation Criteria>

◯: difference between the pH after storage at 50° C. for 2 months and the pH at the time of preparation is within ±0.49
Δ: difference between the pH after storage at 50° C. for 2 months and the pH at the time of preparation is ±0.50 to 0.99
x: difference between the pH after storage at 50° C. for 2 months and the pH at the time of preparation is ±1.0 or more

### (2) Emulsion Stability

Respective emulsion cosmetics of Examples and Comparative Examples were stored at 50° C. for 2 months, and three expert panelists visually observed changes in the emulsion state and the emulsion stability was evaluated based on the discussion of the three expert panelists according to the evaluation criteria below.

### Evaluation Criteria

◯: no change in appearance was observed
Δ: some changes in appearance such as separation of oil, syneresis, and formation of precipitates were observed
x: some changes in appearance such as creaming and formation of precipitates were clearly observed

### (3) Average Particle Size

Respective emulsion cosmetics of Examples 1 to 3 and Comparative Examples 1, 9 were diluted 10-fold with purified water, and the scattering intensity was measured by the dynamic light scattering method using a particle size analyzer for concentrate FPAR-1000 (Otsuka Electronics Co., Ltd.). The average particle size was determined from the scattering intensity distribution.

TABLE 1

| | | Example | | | |
|---|---|---|---|---|---|
| Component name | | 1 | 2 | 3 | 4 |
| 1 | di(phytosteryl/octyldodecyl) | 1.00000 | 1.00000 | 1.00000 | 3.00000 |
| | N-lauroyl-L-glutamate | | | | |
| 2 | hydrogenated lecithin | 0.12000 | 0.12000 | 0.12000 | 0.36000 |
| 3 | decaglyceryl monolaurate | 0.08000 | 0.08000 | 0.08000 | 0.24000 |
| 4 | glycerin | 0.25000 | 0.25000 | 0.25000 | 0.75000 |
| 5 | 1,2-hexanediol | 0.20000 | 0.20000 | 0.20000 | 0.60000 |
| 6 | purified water | 8.35000 | 8.35000 | 8.3\|5000 | 25.05000 |
| 7 | trimethylglycine | 0.26870 | 0.53740 | 1.34350 | 0.80610 |
| 8 | DL-sodium | 0.09750 | 0.19500 | 0.48750 | 0.29250 |
| | pyrrolidonecarboxylate | (0.08330) | (0.16660) | (0.41660) | (0.24990) |
| 9 | L-sodium lactate | 0.05000 | 0.10000 | 0.25000 | 0.15000 |
| | | (0.04020) | (0.08040) | (0.20100) | (0.12100) |
| 10 | L-pyrrolidonecarboxylic acid | 0.03780 | 0.07560 | 0.18900 | 0.11340 |
| 11 | L-serine | 0.01120 | 0.02240 | 0.05600 | 0.03360 |
| 12 | L-alanine | 0.01060 | 0.02120 | 0.05300 | 0.03180 |
| 13 | glycine | 0.01000 | 0.02000 | 0.05000 | 0.03000 |
| 14 | L-glutamic acid | 0.00430 | 0.00860 | 0.02150 | 0.01290 |
| 15 | L-lysine hydrochloride | 0.00340 | 0.00680 | 0.01700 | 0.01020 |
| | | (0.00270) | (0.00540) | (0.01360) | (0.00820) |
| 16 | L-threonine | 0.00320 | 0.00640 | 0.01600 | 0.00960 |
| 17 | L-arginine | 0.01690 | 0.03380 | 0.08450 | 0.05070 |
| 18 | L-proline | 0.00040 | 0.00080 | 0.00200 | 0.00120 |
| 19 | poly(sodiun L-aspartate) | 0.300 | 0.300 | 0.300 | 0.300 |
| | | (0.252) | (0.252) | (0.252) | (0.252) |
| 20 | poly(sodium L-glutamate) | | | | |
| 21 | sodium alginate | | | | |
| 22 | antiseptic | proper | proper | proper | proper |
| | | amount | amount | amount | amount |
| 23 | purified water | amount with total amount as 100 | | | |
| evaluation | pH on preparation | 6.05 | 6.02 | 6.00 | 6.33 |
| item | pH after storage at 50° C. | 6.15 | 6.14 | 6.16 | 6.17 |
| | for 2 months | | | | |
| | pH stability | ◯ | ◯ | ◯ | ◯ |
| | emulsion stability | ◯ | ◯ | ◯ | ◯ |
| | average particle size (nm) | 117.4 | 118.8 | 168.7 | |

| | | Example | | | | |
|---|---|---|---|---|---|---|
| Component name | | 5 | 6 | 7 | 8 | 9 |
| 1 | di(phytosteryl/octyldodecyl) | 3.00000 | 1.00000 | 3.00000 | 5.00000 | 1.00000 |
| | N-lauroyl-L-glutamate | | | | | |
| 2 | hydrogenated lecithin | 0.36000 | 0.12000 | 0.36000 | 0.60000 | 0.12000 |
| 3 | decaglyceryl monolaurate | 0.24000 | 0.08000 | 0.24000 | 0.40000 | 0.08000 |
| 4 | glycerin | 0.75000 | 0.25000 | 0.75000 | 1.25000 | 0.25000 |
| 5 | 1,2-hexanediol | 0.80000 | 0.20000 | 0.60000 | 1.00000 | 0.20000 |
| 6 | purified water | 25.05000 | 8.35000 | 25.05000 | 41.75000 | 8.35000 |
| 7 | trimethylglycine | 1.34350 | 0.26870 | 0.26870 | 0.26870 | 0.80610 |
| 8 | DL-sodium | 0.48750 | 0.09750 | 0.09750 | 0.09750 | 0.29250 |
| | pyrrolidonecarboxylate | (0.41660) | (0.08330) | (0.08330) | (0.08330) | (0.24990) |
| 9 | L-sodium lactate | 0.25000 | 0.05000 | 0.05000 | 0.05000 | 0.15000 |
| | | (0.2 [symbol] 100) | (0.04020) | (0.04020) | (0.04020) | (0.12100) |
| 10 | L-pyrrolidonecarboxylic acid | 0.18900 | 0.03780 | 0.03780 | 0.03780 | 0.11340 |
| 11 | L-serine | 0.05600 | 0.01120 | 0.01120 | 0.01120 | 0.03360 |
| 12 | L-alanine | 0.05300 | 0.01060 | 0.01060 | 0.01060 | 0.03180 |
| 13 | glycine | 0.05000 | 0.01000 | 0.01000 | 0.01000 | 0.03000 |
| 14 | L-glutamic acid | 0.02150 | 0.00430 | 0.00430 | 0.00430 | 0.01290 |
| 15 | L-lysine hydrochloride | 0.01700 | 0.00340 | 0.00340 | 0.00340 | 0.01020 |
| | | (0.01360) | (0.00270) | (0.00270) | (0.00270) | (0.00820) |
| 16 | L-threonine | 0.01600 | 0.00320 | 0.00320 | 0.00320 | 0.00960 |
| 17 | L-arginine | 0.08450 | 0.01690 | 0.01690 | 0.01690 | 0.05070 |
| 18 | L-proline | 0.00200 | 0.00040 | 0.00040 | 0.00040 | 0.00120 |
| 19 | poly(sodiun L-aspartate) | 0.300 | 0.300 | 0.300 | 0.300 | 0.300 |
| | | (0.252) | (0.252) | (0.252) | (0.252) | (0.252) |
| 20 | poly(sodium L-glutamate) | | | | | |
| 21 | sodium alginate | | | | | |
| 22 | antiseptic | proper | proper | proper | proper | proper |
| | | amount | amount | amount | amount | amount |
| 23 | purified water | amount with total amount as 100 | | | | |
| evaluation | pH on preparation | 6.38 | 5.87 | 5.83 | 5.81 | 4.83 |
| item | pH after storage at 50° C. | 6.21 | 5.90 | 5.79 | 5.67 | 4.83 |
| | for 2 months | | | | | |
| | pH stability | ◯ | ◯ | ◯ | ◯ | ◯ |
| | emulsion stability | ◯ | ◯ | ◯ | ◯ | ◯ |
| | average particle size (nm) | | | | | |

| | | Example |
|---|---|---|
| Component name | | 10 |
| 1 | di(phytosteryl/octyldodecyl) | 3.00000 |
| | N-lauroyl-L-glutamate | |
| 2 | hydrogenated lecithin | 0.36000 |
| 3 | decaglyceryl monolaurate | 0.24000 |
| 4 | glycerin | 0.75000 |
| 5 | 1,2-hexanediol | 0.60000 |
| 6 | purified water | 25.05000 |
| 7 | trimethylglycine | 0.80610 |
| 8 | DL-sodium | 0.29250 |
| | pyrrolidonecarboxylate | (0.24990) |
| 9 | L-sodium lactate | 0.15000 |
| | | (0.12100) |
| 10 | L-pyrrolidonecarboxylic acid | 0.11340 |
| 11 | L-serine | 0.03360 |
| 12 | L-alanine | 0.03180 |
| 13 | glycine | 0.03000 |
| 14 | L-glutamic acid | 0.01290 |
| 15 | L-lysine hydrochloride | 0.01020 |
| | | (0.00820) |
| 16 | L-threonine | 0.00960 |
| 17 | L-arginine | 0.05070 |
| 18 | L-proline | 0.00120 |
| 19 | poly(sodiun L-aspartate) | 0.300 |
| | | (0.252) |
| 20 | poly(sodium L-glutamate) | |
| 21 | sodium alginate | |
| 22 | antiseptic | proper |
| | | amount |
| 23 | purified water | amount with total amount as 100 |
| evaluation | pH on preparation | 4.85 |
| item | pH after storage at 50° C. | 4.80 |
| | for 2 months | |
| | pH stability | ◯ |
| | emulsion stability | ◯ |
| | average particle size (nm) | |

| | | Comparative Example | | | | | | | | |
|---|---|---|---|---|---|---|---|---|---|---|
| component name | | 1 | 2 | 3 | 4 | 5 | 6 | 7 | 8 | 9 |
| 1 | di(phytosteryl/octyldodecyl) | 1.00000 | 10.00000 | 1.00000 | 3.00000 | 5.00000 | 8.00000 | 1.00000 | 1.00000 | 10.00000 |
| | N-lauroyl-L-glutamate | | | | | | | | | |
| 2 | hydrogenated lecithin | 0.12000 | 1.20000 | 0.12000 | 0.36000 | 0.60000 | 0.96000 | 0.12000 | 0.12000 | 1.20000 |
| 3 | decaglyceryl monolaurate | 0.08000 | 0.80000 | 0.08000 | 0.24000 | 0.40000 | 0.64000 | 0.08000 | 0.08000 | 0.80000 |
| 4 | glycerin | 0.25000 | 2.50000 | 0.25000 | 0.75000 | 1.25000 | 2.00000 | 0.25000 | 0.25000 | 2.50000 |
| 5 | 1,2-hexanediol | 0.20000 | 2.00000 | 0.20000 | 0.60000 | 1.00000 | 1.60000 | 0.20000 | 0.20000 | 2.00000 |
| 6 | purified water | 8.35000 | 83.50000 | 8.35000 | 25.05000 | 41.75000 | 66.80000 | 8.35000 | 8.35000 | 83.50000 |
| 7 | trimethylglycine | | | 0.26870 | 0.26870 | 0.26870 | 0.26870 | 0.26870 | 0.26870 | |
| 8 | DL-sodium | | | 0.09750 | 0.09750 | 0.09750 | 0.09750 | 0.09750 | 0.09750 | |
| | pyrrolidonecarboxylate | | | (0.08330) | (0.08330) | (0.08330) | (0.08330) | (0.08330) | (0.08330) | |
| 9 | L-sodium lactate | | | 0.05000 | 0.05000 | 0.05000 | 0.05000 | 0.05000 | 0.05000 | |
| | | | | (0.04020) | (0.04020) | (0.04020) | (0.04020) | (0.04020) | (0.04020) | |
| 10 | L-pyrrolidonecarboxylic acid | | | 0.03780 | 0.03780 | 0.03780 | 0.03780 | 0.03780 | 0.03780 | |
| 22 | L-serine | | | 0.01120 | 0.01120 | 0.01120 | 0.01120 | 0.01120 | 0.01120 | |
| 12 | L-alanine | | | 0.01060 | 0.01060 | 0.01060 | 0.01060 | 0.01060 | 0.01060 | |
| 13 | glycine | | | 0.01000 | 0.01000 | 0.01000 | 0.01000 | 0.01000 | 0.01000 | |
| 14 | L-glutamic acid | | | 0.00430 | 0.00430 | 0.00430 | 0.00430 | 0.00430 | 0.00430 | |
| 15 | L-lysine hydrochloride | | | 0.00340 | 0.00340 | 0.00340 | 0.00340 | 0.00340 | 0.00340 | |
| | | | | (0.00270) | (0.00270) | (0.00270) | (0.00270) | (0.00270) | (0.00270) | |
| 16 | L-threonine | | | 0.00320 | 0.00320 | 0.00320 | 0.00320 | 0.00320 | 0.00320 | |
| 17 | L-arginine | | | 0.01690 | 0.01690 | 0.01690 | 0.01690 | 0.01690 | 0.01690 | |
| 18 | L-proline | | | 0.00040 | 0.00040 | 0.00040 | 0.00040 | 0.00040 | 0.00040 | |
| 19 | poly(sodium L-aspartate) | | | | | | | | | |
| 20 | Poly(sodium L-glutamate) | | | | | | | 0.3 | | |
| 21 | sodium alginate | | | | | | | | 0.3 | |
| 22 | antiseptic | proper | proper | proper | proper | proper | proper | proper | proper | proper |
| | | amount | amount | amount | amount | amount | amount | amount | amount | amount |
| 23 | purified water | amount with total amount as 100 | | | | | | | | |
| evaluation | pH on preparation | 5.83 | 5.71 | 6.30 | 6.31 | 6.23 | 6.22 | 6.04 | 6.01 | — |
| item | pH after storage at 50° C. | 3.69 | 3.75. | 5.57 | 5.35 | 5.23 | 5.10 | 5.81 | 5.41 | — |
| | for 2 months | | | | | | | | | |
| | pH stability | x | x | Δ | Δ | x | x | ◯ | Δ | — |
| | emulsion stability | ◯ | ◯ | ◯ | ◯ | ◯ | x | x | x | x |
| | average particle size (nm) | 107 | | | | | | | | 892.4 |

* The numerical value in column corresponding to each component in Table shows content (wt %) of the component, and blank column shows absence of the component. Also, the numerical value in parentheses shows content when converted to a free form.
** In table, “—” means pH was not measured. [symbol]
indicates data missing or illegible when filed

As shown in Table 1, respective emulsion cosmetics of Examples 1 to 10 containing components (a) to (d), and polyhydric alcohol, trimethylglycine, pyrrolidonecarboxylic acid and a salt thereof, lactate, amino acid and amino acid salt as component (e) exhibited good emulsion stability and good pH stability.

Furthermore, the average particle size of the emulsion particles in respective emulsion cosmetics of Examples 1 to 3 was measured and found to be 117.4 nm to 168.7 nm, which confirms that they were fine emulsions.

On the other hand, respective emulsion cosmetics of Comparative Examples 1 and 2 containing component (a) to component (c), and polyhydric alcohol as component (e), but not containing component (d) showed large changes in the pH after storage at 50° C. for 2 months, and the pH stability was found to be poor.

In respective emulsion cosmetics of Comparative Examples 3 to 6 containing component (a) to component (c), and polyhydric alcohol, trimethylglycine, pyrrolidonecarboxylic acid and a salt thereof, lactate, amino acid and amino acid salt as component (e), but not containing component (d), good pH stability was not observed, and as the amount of water added during high-pressure emulsification increased, the pH stability and emulsion stability tended to decrease.

In the emulsion cosmetic of Comparative Example 7 containing component (a) to component (c), and polyhydric alcohol, trimethylglycine, pyrrolidonecarboxylic acid and a salt thereof, lactate, amino acid and amino acid salt as component (e), and containing poly(sodium L-glutamate) instead of component (d), the emulsion stability was poor, and in the emulsion cosmetic of Comparative Example 8 containing sodium alginate instead of component (d), the emulsion stability was poor and a slight decrease in the pH stability was also observed.

In addition, the average particle size of the emulsion particles in the emulsion cosmetic of Comparative Example 1 was measured and it was found to be a fine emulsion composition. However, as mentioned above, the emulsion cosmetic of Comparative Example 1 lacked pH stability.

On the other hand, in the emulsion cosmetic of Comparative Example 9, which was prepared by emulsifying with a normal emulsification equipment without emulsifying with a high-pressure emulsification equipment, the average particle size of the emulsion particles was measured and found to be 892.4 nm. No formation of fine emulsion particles was observed, and emulsion stability was not found.

From the above results, in the emulsion cosmetics containing component (d) to component (c), and the emulsion cosmetics further containing component (e), it was suggested that addition of component (d) improved pH stability and emulsion stability.

It was also suggested that by employing emulsification by a high-pressure emulsification equipment as a means of homogenizing emulsification, a fine emulsion with an average particle size of about 100 nm to about 200 nm can be obtained.

### INDUSTRIAL APPLICABILITY

As described in detail above, the present invention can provide an emulsion composition containing an amino acid-based oil agent, which is superior in emulsion stability and pH stability can be provided, and further, a fine emulsion composition containing an amino acid-based oil agent, which is superior in emulsion stability and pH stability and which can penetrate between the cells of the stratum corneum layer of the skin.

The emulsion composition provided by the present invention can impart the skin with a good moisturizing function and a good barrier function.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An emulsion composition comprising:
(a) at least one amino acid-based oil agent,
(b) lecithin,
(c) at least one polyglycerin fatty acid ester, and
(d) one or more kinds selected from the group consisting of polyaspartic acid and a salt thereof, and having a pH of 4.5 to 7.

2. The emulsion composition according to claim 1, wherein the (a) at least one amino acid-based oil agent is an ester of N-acylamino acid.

3. The emulsion composition according to claim 1, wherein the (b) lecithin is hydrogenated lecithin.

4. The emulsion composition according to claim 1, wherein the (c) at least one polyglycerin fatty acid ester is a polyglycerin fatty acid ester with an HLB value of 14 to 17.

5. The emulsion composition according to claim 1, further comprising (e) at least one water-soluble skin conditioning agent.

6. The emulsion composition according to claim 5, wherein the (e) at least one water-soluble skin conditioning agent is one or more kinds selected from the group consisting of polyhydric alcohol, trimethylglycine, lactic acid and a salt thereof, pyrrolidonecarboxylic acid and a salt thereof, and amino acid and a salt thereof.

7. The emulsion composition according to claim 1, comprising emulsion particles with an average particle size of 30 nm to 500 nm.

8. A method for producing an emulsion composition, comprising uniformly mixing an oil phase component comprising:
(a) at least one amino acid-based oil agent,
(b) lecithin, and
(c) at least one polyglycerin fatty acid ester,
adding an aqueous phase component comprising
(d) one or more kinds selected from the group consisting of polyaspartic acid and a salt thereof to the aforementioned oil phase component, and mixing them, to obtain a mixture, and
emulsifying under high-pressure the mixture, wherein emulsion particles have an average particle size of 30 nm to 500 nm.

9. The production method according to claim 8, wherein the (a) at least one amino acid-based oil agent is an ester of N-acylamino acid.

10. The production method according to claim 8, wherein the (b) lecithin is hydrogenated lecithin.

11. The production method according to claim 8, wherein the (c) at least one polyglycerin fatty acid ester is a polyglycerin fatty acid ester with an HLB value of 14 to 17.

12. The production method according to claim 8, further comprising adding (e) at least one water-soluble skin conditioning agent to the aqueous phase component.

13. The production method according to claim 12, wherein the (e) at least one water-soluble skin conditioning agent is one or more kinds selected from the group consisting of polyhydric alcohol, trimethylglycine, lactic acid and a salt thereof, pyrrolidonecarboxylic acid and a salt thereof, and amino acid and a salt thereof.

14. The production method according to claim 8, comprising setting the pH of the emulsion composition to 4.5 to 7.

15. A method for producing an emulsion composition, comprising adding a part of water to an oil phase component comprising:
(a) at least one amino acid-based oil agent,
(b) lecithin, and
(c) at least one polyglycerin fatty acid ester, and mixing to emulsify them under high pressure, to obtain an emulsion, and
mixing (d) one or more kinds selected from the group consisting of polyaspartic acid and a salt thereof with the rest of water and adding the mixture to the emulsion, wherein emulsion particles have an average particle size of 30 nm to 500 nm.

16. The production method according to claim 15, wherein the (a) at least one amino acid-based oil agent is an ester of N-acylamino acid.

17. The production method according to claim 15, wherein the (b) lecithin is hydrogenated lecithin.

18. The production method according to claim 15, wherein the (c) at least one polyglycerin fatty acid ester is a polyglycerin fatty acid ester with an HLB value of 14 to 17.

19. The production method according to claim 15, further comprising adding at least one polyhydric alcohol as the (e) water-soluble skin conditioning agent to a part of water to be added to the oil phase component.

20. The production method according to claim 15, wherein one or more kinds selected from the group consisting of trimethylglycine, lactic acid and a salt thereof, pyrrolidonecarboxylic acid and a salt thereof, and amino acid and a salt thereof are added as the (e) water-soluble skin conditioning agent to the rest of water and mixed with the emulsion.

21. The production method according to claim 15, comprising setting the pH of the emulsion composition to 4.5 to 7.
